(19) United States
(12) Patent Application Publication
(10) Pub. No.: US 2022/0002653 A1
(43) Pub. Date: Jan. 6, 2022

(54) CELL THERAPY VESSELS

(71) Applicant: bluebird bio, Inc., Cambridge, MA (US)

(72) Inventor: ALDEN LADD, CAMBRIDGE, MA (US)

(73) Assignee: bluebird bio, Inc., Cambridge, MA (US)

(21) Appl. No.: 17/291,323

(22) PCT Filed: Nov. 6, 2019
(86) PCT No.: PCT/US2019/059991

§ 371 Date: May 5, 2021

Related U.S. Application Data
(60) Provisional application No. 62/756,816, filed on Nov. 7, 2018.

(51) Int. Cl. C12M 1/26; C12M 1/00; C12M 3/00

(52) U.S. Cl. CPC C12M 33/10; C12M 23/14; C12M 29/00; C12M 23/50; C12M 23/48; C12M 47/02

(57) ABSTRACT

The present disclosure provides improved cell therapy vessels, cell therapy vessel adapters, kits, and methods of using the same.

### CROSS REFERENCE TO RELATED APPLICATIONS

This application claims the benefit under 35 U.S.C. § 119(e) of U.S. Provisional Application No. 62/756,816, filed Nov. 7, 2018, which is incorporated by reference herein in its entirety.

### BACKGROUND

### Technical Field

The present disclosure relates to improved cell processing vessels. More particularly, the disclosure relates to improved vessels for manufacturing cell and gene therapies.

### Description of the Related Art

Cell and gene therapies bring tremendous promise to patients, potentially offering one-time curative therapies. However, the process of manufacturing such therapies is complex and expensive. Potential bottlenecks and inefficiencies in cell and gene therapy manufacturing processes threaten the delivery of these promising therapies to patients.

Currently, the art lacks efficient closed system solutions to exchange reagents, formulate, and/or concentrate cells. Existing solutions rely on filtration technology and require performing complex processing steps, opening up the manufacturing system, and/or relying on complex machinery to perform tasks that require complete media exchange, formulation, and/or concentrating cell and gene therapies. The inability to reliably process cell and gene therapies drives up the cost and increases the complexity and variability of the manufacturing process. In the absence of efficient cell processing solutions, the cost to manufacture cell and gene therapies will continue to be high, as will the regulatory hurdles needed to approve and deliver these therapies to patients.

### BRIEF SUMMARY

The present disclosure generally relates, in part, to improved cell therapy vessels. More particularly, the disclosure relates to improved cell therapy vessels for cell and gene therapies and methods of using the same in closed manufacturing processes.

In various embodiments, a cell therapy vessel is provided comprising a first intake/output line; a body comprising a rectangular portion and a conical portion; a collection reservoir; and a second intake/output line.

In particular embodiments, the vessel comprises one or more ports.

In particular embodiments, the vessel comprises one port on the top of the vessel body and one port on the bottom of the collection reservoir.

In some embodiments, the first intake/output line is connected to the top of the vessel body.

In certain embodiments, the first intake/output line is connected to the port on the top of the vessel body.

In additional embodiments, the first intake/output line is welded to the top of the vessel body.

In particular embodiments, the rectangular portion has a width of about 4 inches to about 6 inches.

In further embodiments, the rectangular portion has a width of about 5 inches.

In some embodiments, the conical portion has a maximum width of about 4 inches to about 6 inches and a minimum width of about 1 inch to about 0.5 inches.

In particular embodiments, the conical portion has a maximum width of about 5 inches and a minimum width of about 0.75 inches.

In additional embodiments, the collection reservoir has a width of about 1 inch to about 0.5 inches.

In particular embodiments, the second intake/output line is connected to the bottom of the collection reservoir.

In some embodiments, the second intake/output line is connected to the port on the bottom of the collection reservoir.

In certain embodiments, the second intake/output line is welded to the bottom of the collection reservoir.

In further embodiments, the length of the body of the cell therapy vessel is about 5 inches to about 7 inches.

In particular embodiments, the length of the body of the cell therapy vessel is about 6 inches.

In some embodiments, the body of the cell therapy vessel has a volume of about 300 mLs to about 600 mLs.

In particular embodiments, the body of the cell therapy vessel has a volume of about 300, 301, 302, 303, 304, 305, 306, 307, 308, 309, 310, 311, 312, 313, 314, 315, 316, 317,318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417,318, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517,318, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or about 600 mLs.

In certain embodiments, the body of the cell therapy vessel has a volume of about 400 mLs.

In additional embodiments, the body of the cell therapy vessel has a volume of about 500 mLs.

In particular embodiments, the volume of the rectangular portion and the conical portion of the cell therapy vessel is about 15-fold to about 25-fold of the volume of the collection reservoir.

In additional embodiments, the volume of the rectangular portion and the conical portion of the cell therapy vessel is about 20-fold of the volume of the collection reservoir.

In some embodiments, the volume of the collection reservoir is about 1% to about 10% of the volume of the rectangular portion and the conical portion of the cell therapy vessel.

In particular embodiments, the volume of the collection reservoir is about 5% of the volume of the rectangular portion and the conical portion of the cell therapy vessel.

In particular embodiments, the cell therapy vessel is comprised of one or more materials selected from the group consisting of: polyvinylchloride, polyethylene, polypropylene, and fluorinated ethylene propylene.

In various embodiments, a cell therapy vessel is provided comprising: (a) a first intake/output line fused to the top of the vessel; (b) a rectangular portion; (c) a conical portion; (d) a cell collection reservoir; and (e) a second intake/output line fused to the bottom of the cell collection reservoir.

In additional embodiments, the vessel further comprises one or more ports.

In certain embodiments, the rectangular portion has a width of about 4 inches to about 6 inches.

In certain embodiments, the rectangular portion has a width of about 5 inches.

In further embodiments, the conical portion has a maximum width of about 4 inches to about 6 inches and a minimum width of about 1 inch to about 0.5 inches.

In further embodiments, the conical portion has a maximum width of about 5 inches and a minimum width of about 0.75 inches.

In particular embodiments, the collection reservoir has a width of about 1 inch to about 0.5 inches.

In particular embodiments, the length of the body of the cell therapy vessel is about 5 inches to about 7 inches.

In some embodiments, the length of the body of the cell therapy vessel is about 6 inches.

In additional embodiments, the body of the cell therapy vessel has a volume of about 300 mLs to about 600 mLs.

In certain embodiments, the body of the cell therapy vessel has a volume of about 300, 301, 302, 303, 304, 305, 306, 307, 308, 309, 310, 311, 312, 313, 314, 315, 316, 317,318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417,318, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517,318, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or about 600 mLs.

In particular embodiments, the body of the cell therapy vessel has a volume of about 400 mLs.

In additional embodiments, the body of the cell therapy vessel has a volume of about 500 mLs.

In some embodiments, the volume of the rectangular portion and the conical portion of the cell therapy vessel is about 15-fold to about 25-fold of the volume of the collection reservoir.

In particular embodiments, the volume of the rectangular portion and the conical portion of the cell therapy vessel is about 20-fold of the volume of the collection reservoir.

In certain embodiments, the volume of the collection reservoir is about 1% to about 10% of the volume of the rectangular portion and the conical portion of the cell therapy vessel.

In particular embodiments, the volume of the collection reservoir is about 5% of the volume of the rectangular portion and the conical portion of the cell therapy vessel.

In further embodiments, the cell therapy vessel is a bag comprised of one or more materials selected from the group consisting of: polyvinylchloride, polyethylene, polypropylene, and fluorinated ethylene propylene.

In some embodiments, the cell therapy vessel contains a population of cells.

In particular embodiments, the population of cells is genetically modified.

In some embodiments, the population of cells comprises one or more genome edits.

In additional embodiments, the population of cells comprises a gene therapy vector.

In particular embodiments, the gene therapy vector encodes a therapeutic protein.

In further embodiments, the gene therapy vector encodes a chimeric antigen receptor (CAR) or engineered T cell receptor (TCR).

In certain embodiments, the population of cells is obtained from peripheral blood mononuclear cells, bone marrow, lymph nodes tissue, cord blood, thymus issue, tissue from a site of infection, ascites, pleural effusion, spleen tissue, or tumors.

In further embodiments, the population of cells comprises hematopoietic stem or progenitor cells.

In particular embodiments, the population of cells comprises CD34+ cells.

In some embodiments, the population of cells comprises lymphocytes.

In certain embodiments, the population of cells comprises T cells.

In certain embodiments, the population of cells comprises CD3+, CD4+, and/or CD8+ T cells.

In particular embodiments, the population of cells comprises effector cells.

In additional embodiments, the population of cells comprises cytotoxic T lymphocytes (CTLs), tumor infiltrating lymphocytes (TILs), or helper T cells.

In additional embodiments, the population of cells comprises natural killer (NK) cells or natural killer T (NKT) cells.

In further embodiments, the cell therapy vessel is a bag that comprises one or more materials selected from the group consisting of: polyvinylchloride, polyethylene, polypropylene, and fluorinated ethylene propylene.

In particular embodiments, the cell therapy vessel is endotoxin free.

In various embodiments, a cell therapy vessel adapter is provided comprising: (a) an inner portion shaped to receive the cell therapy vessel contemplated herein; (b) an outer portion shaped to fit into a centrifuge; wherein the outer portion further comprises an opening for the second intake/output line of the cell therapy vessel and a groove to receive the second intake/output line.

In certain embodiments, the cell therapy vessel adapter comprises one or more materials selected from the group consisting of: polyvinylchloride, polyethylene, polypropylene, and fluorinated ethylene propylene.

In various embodiments, a kit is provided comprising: (a) a cell therapy vessel, and (b) a cell therapy vessel adapter.

In particular embodiments, the kit further comprises one or more clamps.

In various embodiments, a method for processing a cell therapy product is provided comprising introducing a population of cells into the cell therapy vessel; placing the cell therapy vessel into the cell therapy vessel adapter, and centrifuging the population of cells under conditions sufficient to concentrate the population of cells in the cell collection reservoir of the cell therapy vessel.

In further embodiments, processing comprises performing one or more processing steps selected from the group consisting of: concentrating the cells, exchanging cell culture medium or buffer, washing the cells, genetically modifying the cells, preparing the cells for electroporation, preparing the cells for transduction, transducing the cells, formulating the cells for administration, or formulating the cells for cryopreservation.

In particular embodiments, the population of cells is genetically modified.

In some embodiments, the population of cells is concentrated by centrifugation after one or more processing steps.

In certain embodiments, processing comprises introducing one or more materials into the cell therapy vessel through the one or more ports or the first or second intake/output line.

In additional embodiments, processing comprises removing one or more materials from the cell therapy vessel through the one or more ports or the first or second intake/output line.

In particular embodiments, the population of cells is introduced to the cell therapy vessel through the first intake/output line.

In certain embodiments, the population of cells is removed from the cell therapy vessel through the first intake/output line.

In various embodiments, a method for manufacturing a cell-based therapy is provided comprising introducing a population of cells into the cell therapy vessel; placing the cell therapy vessel into the cell therapy vessel adapter, and centrifuging the population of cells under conditions sufficient to concentrate the population of cells in the cell collection reservoir of the cell therapy vessel.

In particular embodiments, the cell-based therapy is an adoptive cellular therapy, a cell-based gene therapy, or a genome-edited cell therapy.

In some embodiments, the population of cells is genetically modified before introduction into the vessel.

In some embodiments, the population of cells is genetically modified in the vessel.

In particular embodiments, the cell-based therapy is CAR T cell therapy.

In further embodiments, the cell-based therapy is a gene therapy for hemoglobinopathies.

In certain embodiments, the cell-based therapy is a gene therapy for adrenoleukodystrophies.

In particular embodiments, the cell-based therapy is a genome-edited cell therapy for a cancer.

In additional embodiments, processing comprises performing one or more processing steps selected from the group consisting of: concentrating the cells, exchanging cell culture medium or buffer, washing the cells, genetically modifying the cells, preparing the cells for electroporation, preparing the cells for transduction, transducing the cells, formulating the cells for administration, or formulating the cells for cryopreservation.

In particular embodiments, the population of cells is concentrated by centrifugation after one or more processing steps.

In some embodiments, processing comprises introducing one or more materials into the cell therapy vessel through the one or more ports or the first or second intake/output line.

In certain embodiments, processing comprises removing one or more materials from the cell therapy vessel through the one or more ports or the first or second intake/output line.

In particular embodiments, the population of cells is introduced to the cell therapy vessel through the first intake/output line.

In particular embodiments, the population of cells is removed from the cell therapy vessel through the first intake/output line.

### BRIEF DESCRIPTION OF SEVERAL VIEWS OF THE DRAWINGS

FIG. 1 shows a representative diagram of a front view of a cell therapy vessel.

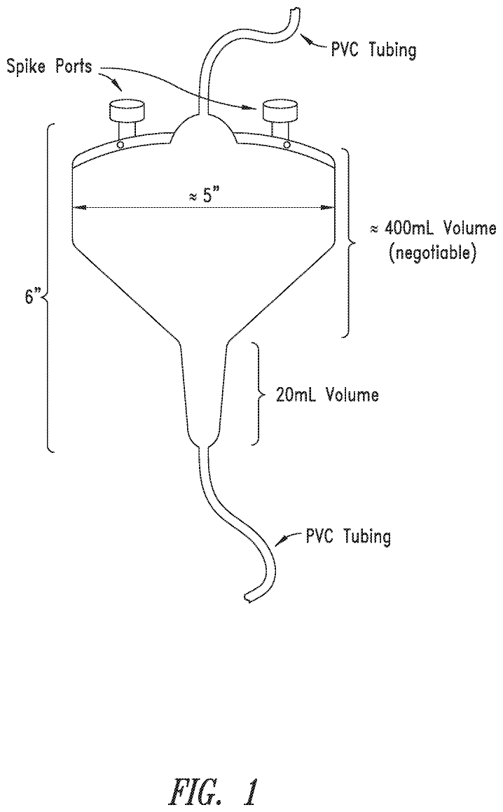

FIG. 2 shows a representative diagram of a front view of a cell therapy vessel.

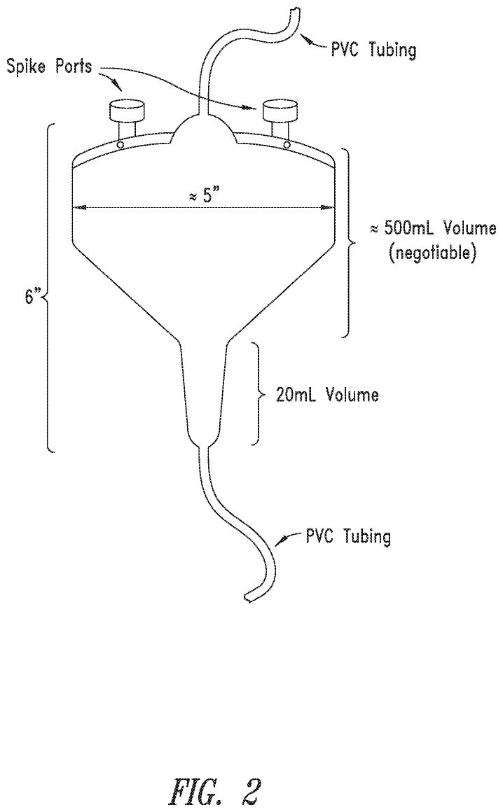

FIG. 3 shows a representative diagram of a side view of a cell therapy vessel.

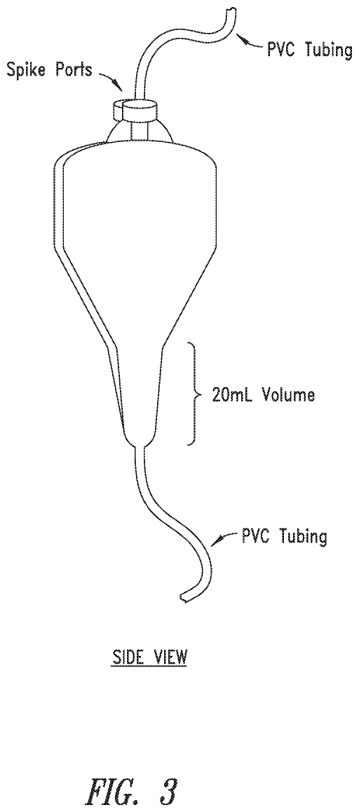

FIG. 4 shows a diagram of a side view of a cell therapy vessel adapter.

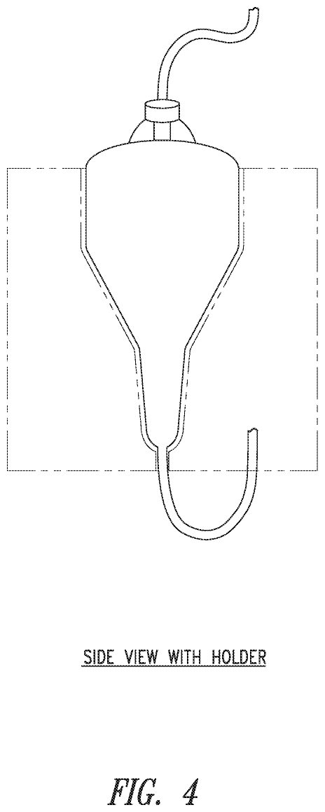

### DETAILED DESCRIPTION

### A. Overview

The present disclosure generally relates to, in part, improved vessels for processing cellular compositions. In particular, the vessels contemplated herein provide solutions to various problems encountered in manufacturing therapeutic cell compositions.

The lack of efficient, robust, and reliable cell processing vessels places significant limitations on the development of cost-effective closed system manufacturing processes. Closed systems require performing numerous operations on cellular compositions including, but not limited to concentrating cells, performing complete medium exchanges, washing cells, preparing cells for electroporation, preparing cells for transduction, transducing cells, formulating cells for administration, and formulating cells for cryopreservation. Current solutions posed to closed system manufacturing problems are ineffective, rigid, time-consuming, and expensive to implement; the solutions rely on filtration technology and often require performing complex processing steps, opening up the manufacturing system, and/or relying on complex machinery to perform a variety of cell processing. The inability of the art to solve the problem of providing flexible and reliable cell processing vessels drives up the cost, increases the complexity and variability of these manufacturing processes, and jeopardizes the delivery of one-time potentially curative therapies to patients.

The present invention provides efficient, robust, reliable, and cost-effective solutions for cell processing in cell therapy manufacturing processes. In particular embodiments, the manufacturing process is an open system. In preferred embodiments, the manufacturing process is a closed system.

In various embodiments, a cell therapy vessel is provided. In particular embodiments, the cell therapy vessel is suitable for use in a closed system manufacturing process of cell therapy. Without wishing to be bound by any particular theory, cell therapy vessels contemplated herein are believed to enable an efficient, reliable, robust, low-cost solution to the manufacturing problems related to cell processing. The vessels contemplated in preferred embodiments are designed so as to enable various cell processing operations in the same vessel and to allow concentration and retrieval of the processed cells.

In various embodiments, a cell therapy vessel is provided for use in a closed system manufacturing process of cell therapy, e.g., adoptive cellular therapy, cell-based gene therapy, or genome-edited cell therapy. In preferred embodiments, the cell therapy vessel comprises a cell collection reservoir that allows efficient concentration and reliable retrieval of processed cells.

In various embodiments, an adapter that is designed to hold a cell therapy vessel contemplated herein is provided. The vessel and adapter can be provided separately or together in a kit. Without wishing to be bound by any particular theory, it is contemplated that a cell therapy vessel placed inside a cell therapy vessel adaptor allows for cellular processing in many types of centrifuges, further enabling a simplified, low-cost solution to complex manufacturing problems.

In various embodiments, a method of processing a population of cells is provided. In particular embodiments, a method for processing cells comprises introducing a population of cells into a vessel and performing one or more cellular processing steps. In particular embodiments, the method further comprises putting the vessel into an adapter and concentrating the population of cells by centrifugation. In preferred embodiments, a method for processing cells comprises introducing a population of cells into a vessel, performing a cellular processing step, concentrating the cells by centrifugation, and repeating the process using one or more of the same or different processing steps.

In various embodiments, a method of manufacturing a cell therapy product is provided. In particular embodiments, a method of manufacturing a cell therapy comprises introducing a population of therapeutic cells into a vessel and performing one or more cellular processing steps, optionally including genetic modification of the therapeutic cells. In preferred embodiments, the method further comprises putting the vessel into an adapter and concentrating the cell therapy product by centrifugation. In preferred embodiments, a method for processing cells comprises introducing a population of cells into a vessel, performing a cellular processing step, optionally including genetic modification of the therapeutic cells, concentrating the cells by centrifugation, and repeating the process using one or more of the same or different processing steps.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, N.Y.); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir andCC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

It is also understood that the positive recitation of a feature in one embodiment, serves as a basis for excluding the feature in a particular embodiment.

A “closed system” refers to a system that automates one or more cell processing steps including, but not limited to, media exchange, cell culture, treatment, centrifugation, incubation, media addition, cell selection, cell washing, formulation for cryopreservation, and formulation for administration.

It is to be understood that the terms “top”, “bottom”, “upper”, “lower”, “right”, “left”, “vertical”, “horizontal”, “length”, “width”, “height”, “thickness”, “front”, “back”, “rear”, “side” and the like are used herein merely to describe points of reference and do not limit the present invention to any specific configuration or orientation.

### C. Cell Therapy Vessels

Improved vessels for processing cells are contemplated herein. In preferred embodiments, the vessels contemplated herein are cell therapy vessels that are suitable for performing one or more cell processing steps in the same vessel. In preferred embodiments, the vessels contemplated herein are cell therapy vessels that are suitable for performing one or more cell processing steps in the same vessel in a closed system manufacturing process. Cell therapy vessels contemplated herein provide substantial advantages compared to existing vessels including, but not limited to, simple low-cost manufacturing of the vessel, reducing cost-of-goods (COGs) for manufacturing processes, increasing efficiency, reliability, and robustness of closed system manufacturing processes, reducing opportunity for introducing contaminants into cell therapy manufacturing processes, and enabling use of single vessel for multiple cellular processing steps.

Cell therapy vessels contemplated in particular embodiments may be in the form of an intravenous (IV) bag, a cell culture bag or a bioreactor bag.

Cell therapy vessels contemplated in particular embodiments are made from materials that comprise one or more of the following characteristics: gas permeability (materials have suitable gas transfer rates for oxygen, carbon dioxide and nitrogen); negligible water loss rates (materials are practically impermeable to water); chemically and biologically inert (materials do not react with the vessel contents), and retention of flexibility and strength in various conditions (materials enable vessel to be microwaved, treated with UV irradiation, centrifuged, or used within a broad range of temperatures, e.g., from −100° C. to +100° C.).

In particular embodiments, a cell therapy vessel is fabricated using polyvinylchloride, polyethylene, polypropylene, or fluorinated ethylene propylene.

In preferred embodiments, a cell therapy vessel is substantially free of mycoplasma, endotoxin, and microbial contamination. By “substantially free” with respect to endotoxin means that a vessel is at least 95%, at least 96%, at least 97%, at least 98%, at least 99%, or 100% endotoxin free.

In preferred embodiments, a cell therapy vessel is manufactured under good manufacturing practice (GMP).

In particular embodiments, a cell therapy vessel comprises a body and a collection reservoir. Without wishing to be bound by any particular theory, the contemplated vessels are believed to be the first vessels with a design that enables multiple processing steps in a closed system and the ability to subsequently retrieve a cell pellet comprising the processed cells.

In particular embodiments, the volume of the body is at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 times the volume of the cell collection reservoir allowing for closed system processing and retrieval of a concentrated population of processed cells.

In particular embodiments, a cell therapy vessel comprises a body that has a volume of about 250 mLs to about 1000 mLs, about 250 mLs to about 900 mLs, about 250 mLs to about 800 mLs, about 250 mLs to about 750 mLs, about 300 mLs to about 600 mLs, about 300 mLs to about 500 mLs, about 400 mLs to about 500 mLs, about 300 mLs to about 500 mLs, or about 350 mLs to about 450 mLs.

In particular embodiments, a cell therapy vessel comprises a body that has a volume of about 250 mLs, about 275 mLs, about 300 mLs, about 325 mLs, about 350 mLs, about 375 mLs, about 400 mLs, about 425 mLs, about 450 mLs, about 475 mLs, about 500 mLs, about 525 mLs, about 550 mLs, about 575 mLs, about 600 mLs, about 625 mLs, about 650 mLs, about 675 mLs, about 700 mLs, about 725 mLs, about 750 mLs, about 775 mLs, about 800 mLs, about 825 mLs, about 850 mLs, about 875 mLs, about 900 mLs, about 925 mLs, about 950 mLs, about 975 mLs, or about 1000 mLs or any intervening volume. In preferred embodiments, a cell therapy vessel has a body of about 400 mLs. In preferred embodiments, a cell therapy vessel has a body of about 500 mLs. In preferred embodiments, a cell therapy vessel has a body of about 400 mLs to about 500 mLs.

In certain embodiments, a cell therapy vessel comprises a body that has a volume of about 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417,318, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517,318, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, or 550 mLs.

In certain embodiments, a cell therapy vessel comprises a body that has a volume of about 400, 401, 402, 403, 404, 405, 406, 407, 408, 409, 410, 411, 412, 413, 414, 415, 416, 417,318, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mLs.

In certain embodiments, a cell therapy vessel comprises a body that has a volume of about 500, 501, 502, 503, 504, 505, 506, 507, 508, 509, 510, 511, 512, 513, 514, 515, 516, 517,318, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or about 600 mLs.

In certain embodiments, a cell therapy vessel comprises a body that has a volume of about 600, 601, 602, 603, 604, 605, 606, 607, 608, 609, 610, 611, 612, 613, 614, 615, 616, 617,318, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, or about 700 mLs. In certain embodiments, a cell therapy vessel comprises a body that has a volume of about 700, 701, 702, 703, 704, 705, 706, 707, 708, 709, 710, 711, 712, 713, 714, 715, 716, 717,318, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, or about 800 mLs.

In certain embodiments, a cell therapy vessel comprises a body that has a volume of about 800, 801, 802, 803, 804, 805, 806, 807, 808, 809, 810, 811, 812, 813, 814, 815, 816, 817,318, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, or about 900 mLs.

In certain embodiments, a cell therapy vessel comprises a body that has a volume of about 900, 901, 902, 903, 904, 905, 906, 907, 908, 909, 910, 911, 912, 913, 914, 915, 916, 917,318, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or about 1000 mLs.

In particular embodiments, the volume of the body is at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 times the volume of the cell collection reservoir allowing for processing and retrieval of a concentrated population of processed cells.

In certain embodiments, the volume of the cell collection reservoir is about 5 mLs, about 10 mLs, about 15 mLs, about 20 mLs, about 25 mLs, about 30 mLs, about 35 mLs, about 40 mLs, about 45 mLs, about 50 mLs, about 55 mLs, about 60 mLs, about 65 mLs, about 70 mLs, about 75 mLs, about 80 mLs, about 85 mLs, about 90 mLs, about 95 mLs, or about 100 mLs.

In particular embodiments, the volume of the cell collection reservoir is about 15 mLs, 16 mLs, 17 mLs, 18 mLs, 19 mLs, 20 mLs, 21 mLs, 22 mLs, 23 mLs, 24 mLs, or 25 mLs,

In particular embodiments, a cell therapy vessel comprises one or more intake/output ports; a body; and a collection reservoir having volumes disclosed herein, e.g., supra, wherein the dimensions of the body and collection reservoir contribute to the processing efficiency of a population of cells.

Cell therapy vessels contemplated in particular embodiments may be so dimensioned as to achieve the desired cell processing volumes and ratios of volumes of the vessel body to the cell reservoir collection.

In particular embodiments, a body of a vessel comprises two or more portions of different dimensions. In a preferred embodiment, a vessel comprises a body having a rectangular portion and a conical portion. In another preferred embodiment, the top of the vessel body comprises a rectangular portion, the bottom of the rectangular portion forming the top of the conical portion of the vessel, the bottom of the conical portion forming the top of the collection reservoir.

In certain embodiments, a vessel comprises a body having a rectangular portion and a conical portion, wherein the rectangular portion has a width of about 4 inches to about 6 inches.

In particular embodiments, a vessel comprises a body having a rectangular portion and a conical portion, wherein the rectangular portion has a width of about 4.0 inches, about 4.1 inches, about 4.2 inches, about 4.3 inches, about 4.4 inches, about 4.5 inches, about 4.6 inches, about 4.7 inches, about 4.8 inches, about 4.9 inches, about 5.0 inches, about 5.1 inches, about 5.2 inches, about 5.3 inches, about 5.4 inches, about 5.5 inches, about 5.6 inches, about 5.7 inches, about 5.8 inches, about 5.9 inches, or about 6.0 inches. In preferred embodiments, a vessel comprises a body having a rectangular portion and a conical portion, wherein the rectangular portion has a width of about 5.0 inches.

In particular embodiments, a vessel comprises a body having a rectangular portion adjoined to a conical portion, wherein the conical portion has a maximum width of about 4 inches to about 6 inches. In particular embodiments, a vessel comprises a body having a rectangular portion and a conical portion, wherein the conical portion has a maximum width of about 4.0 inches, about 4.1 inches, about 4.2 inches, about 4.3 inches, about 4.4 inches, about 4.5 inches, about 4.6 inches, about 4.7 inches, about 4.8 inches, about 4.9 inches, about 5.0 inches, about 5.1 inches, about 5.2 inches, about 5.3 inches, about 5.4 inches, about 5.5 inches, about 5.6 inches, about 5.7 inches, about 5.8 inches, about 5.9 inches, or about 6.0 inches. In preferred embodiments, a vessel comprises a body having a rectangular portion and a conical portion, wherein the conical portion has a maximum width of about 5.0 inches. In certain embodiments, a conical portion of a vessel has a minimum width of about 0.5 inches to about 1.5 inches. In certain embodiments, a conical portion of a vessel has a minimum width of about 0.5 inches, about 0.6 inches, about 0.7 inches, about 0.8 inches, about 0.5 inches, about 1.0 inches, about 1.1 inches, about 1.2 inches, about 1.3 inches, about 1.4 inches, or about 1.5 inches.

In particular embodiments, a vessel comprises a body having a rectangular portion adjoined to a conical portion, the conical portion adjoined to a cell collection reservoir having a width of about 0.5 inches to about 1.5 inches. In certain embodiments, a cell collection reservoir of a vessel has a width of about 0.5 inches, about 0.6 inches, about 0.7 inches, about 0.8 inches, about 0.5 inches, about 1.0 inches, about 1.1 inches, about 1.2 inches, about 1.3 inches, about 1.4 inches, or about 1.5 inches. In certain embodiments, a cell collection reservoir has a length of about 2 to about 3 inches, or about 2.0 inches, about 2.1 inches, about 2.2 inches, about 2.3 inches, about 2.4 inches, about 2.5 inches, about 2.6 inches, about 2.7 inches, about 2.8 inches, about 2.9 inches, about 3.0 inches.

In certain embodiments, a vessel comprises a body and a cell collection reservoir having the dimensions contemplated herein, e.g., supra, wherein the length of the vessel is about 5 inches to about 7 inches. In particular embodiments, a vessel has a length of about 5.0 inches, about 5.1 inches, about 5.2 inches, about 5.3 inches, about 5.4 inches, about 5.5 inches, about 5.6 inches, about 5.7 inches, about 5.8 inches, about 5.9 inches, about 6.0 inches, about 6.1 inches, about 6.2 inches, about 6.3 inches, about 6.4 inches, about 6.5 inches, about 6.6 inches, about 6.7 inches, about 6.8 inches, about 6.9 inches, or about 7.0 inches. In preferred embodiments, a vessel has a length of about 6.0 inches.

In particular embodiments, a vessel is fabricated to have a cross-sectional wall thickness that is between about 0.25 mm and about 2.0 mm, between about 0.5 mm and about 1.5 mm, and between about 0.75 mm and about 1.25 mm In particular embodiments, the cross-sectional wall thickness of a vessels is at least 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm or any intervening thickness.

In particular embodiments, a cell therapy vessel comprises a first intake/output line, a body, a collection reservoir, and a second intake/output line and optionally, one or more intake/output ports. An intake/output line is advantageous in particular embodiments, and in closed systems, because it allows a way to introduce a population of cells and/or other materials or substances into the system. An intake/output line is also advantageous in particular embodiments, and in closed systems, because it allows a way to efficiently remove materials and processed cells from a vessel. In certain embodiments, vessels comprise one or more ports that each independently comprises a valve, a filter, or a combination thereof. In some embodiments, vessels comprise one or more ports that do not comprise a valve, a filter, or a combination thereof. In particular embodiments, a port is resealable. In particular embodiments, a first intake/output line is connected to the top of the vessel. In particular embodiments, a first intake/output line is connected to a port on the top or upper portion of the vessel. In particular embodiments, a first intake/output line is welded to the top or upper portion of the vessel. In particular embodiments, a second intake/output line is connected to the bottom of the vessel. In particular embodiments, a second intake/output line is connected to a port on the bottom or lower portion of the vessel. In particular embodiments, a second intake/output line is welded to the bottom or lower portion of the vessel. In particular embodiments, a vessel comprises one or more intake/output lines connected to a vessel. In certain embodiments, a vessel comprises one or more intake/output lines and/or ports connected to the top of the vessel and one or more intake/output lines and/or ports connected to the bottom of the vessel. In particular embodiments, a vessel comprises one or more intake/output lines connected to one or more ports on the upper portion of the vessel and one or more intake/output lines connected to one or more ports on the bottom of the vessel. In particular embodiments, a vessel comprises one or more ports on the upper and/or lower portions of the vessel and one or more intake/output lines welded to the upper portion of the vessel and one or more intake/output lines welded to the bottom of the vessel.

In particular embodiments, a cell therapy vessel comprises a first intake/output line connected to the top of or upper portion of a vessel body; a vessel body comprising a rectangular portion and a conical portion; a cell collection reservoir; a second intake/output line connected to the bottom or lower portion of the cell collection reservoir; and optionally one or more intake/output ports. In particular embodiments, a first intake/output line is connected to a port on the rectangular portion of the vessel body. In particular embodiments, a first intake/output line is welded to the top of the rectangular portion of the vessel body. In one embodiment, a second intake/output line is connected to a port on the cell collection reservoir of the vessel. In one embodiment, a second intake/output line is welded to the bottom of the cell collection reservoir of the vessel.

In particular embodiments, a vessel comprises one or more intake/output lines connected to a vessel body, the vessel body comprising a rectangular portion and a conical portion, and an intake/output line connected to a cell collection reservoir. In certain embodiments, a vessel comprises one or more intake/output lines and/or ports connected to the rectangular portion of the vessel body and an intake/output line and/or port connected to the cell collection reservoir of the vessel. In particular embodiments, a vessel comprises one or more intake/output lines connected to one or more ports on the rectangular portion of the vessel and an intake/output line connected to a port on the bottom of the cell collection reservoir of the vessel. In particular embodiments, a vessel comprises one or more ports on the upper and/or lower portions of the vessel body, and one or more intake/output lines welded to the rectangular portion of the vessel and an intake/output line welded to the bottom of the cell collection reservoir of the vessel.

In particular embodiments, a vessel comprises at least one line for introducing and/or removing cells and/or other substances to or from the vessel and that is connected to a vessel body, e.g., the top or upper portion of the vessel body, the vessel body comprising a rectangular portion and a conical portion, and at least one line for introducing and/or removing cell and/or other substances to or from the vessel and that is connected to a cell collection reservoir, e.g., the bottom of the cell collection reservoir. In particular embodiments, a vessel comprises at least one line connected to the upper portion or rectangular portion of the vessel body and that is for introducing and/or removing cells and/or other substances to or from the vessel, the vessel body comprising a rectangular portion and a conical portion, and at least one line connected to the cell collection reservoir and that is for introducing and/or removing cell and/or other substances to or from the vessel. In particular embodiments, a vessel comprises at least one line connected to a port on the rectangular portion of the vessel body and that is for introducing and/or removing cells and/or other substances to or from the vessel, the vessel body comprising a rectangular portion and a conical portion, and at least one line connected to a port on the cell collection reservoir and that is for introducing and/or removing cell and/or other substances to or from the vessel. In particular embodiments, a vessel comprises at least one line welded to the top of the rectangular portion of the vessel body and that is for introducing and/or removing cells and/or other substances to or from the vessel, the vessel body comprising a rectangular portion and a conical portion, and at least one line welded to the bottom of the cell collection reservoir and that is for introducing and/or removing cell and/or other substances to or from the vessel.

In particular embodiments, a cell therapy vessel comprises a means for introducing and/or removing cells and/or other substances to or from the vessel, a vessel body, and a means for collecting cells from the vessel.

### D. Cells

Cell therapy vessels contemplated herein are suitable for performing one or more cellular processing steps for manufacturing of cell-based therapies including but not limited to adoptive cell therapies, genome edited cell therapies, and cell-based gene therapies. In preferred embodiments, vessels contemplated herein are suitable for performing one or more cellular processing steps on a population of cells in the same vessel including, but not limited to, one or more partial or complete medium exchanges, concentrating cells, washing cells, preparing cells for electroporation, preparing cells for transduction, transducing cells, formulating cells for administration, or formulating cells for cryopreservation.

In particular embodiments, cell therapy vessels contemplated herein are used to manufacture a cell-based therapy comprising genetically modified cells. In particular embodiments, the genetically modified cells comprise one or more vectors encoding a therapeutic transgene, e.g., a globin, an engineered antigen receptor. Illustrative examples of vectors include, but are not limited to, viral vectors. Illustrative examples of viral vectors include, but are not limited to: an adenovirus, an adeno-associated virus (AAV), a retrovirus, e.g., a lentivirus (e.g., HIV-1, HIV-2), a herpes simplex virus e.g., HSV-1, HSV-2), or a vaccinia virus. In particular embodiments, the genetically modified cells comprise one or more genome edits. In certain embodiments, the genetically modified cells comprise one or more vectors encoding a therapeutic transgene and one or more gene edits.

In particular embodiments, the improved cell therapy vessels contemplated herein are used to manufacture cell-therapies for the prevention, treatment, or amelioration of at least one symptom, of a monogenetic disease, disorder, or condition, e.g., a hemoglobinopathy, cerebral adrenoleukodystrophy, cancer, GVHD, infectious disease, autoimmune disease, immunodeficiency or condition associated therewith.

Cells suitable for manufacturing or processing in the cell therapy vessels contemplated in particular embodiments may be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are obtained from a mammalian subject. In a more preferred embodiment, the cells are obtained from a primate subject. In an even more preferred embodiment, the cells are obtained from a human subject. In another preferred embodiment, the cells are obtained from a human subject that will be treated with the cell-based therapy.

An “isolated cell” refers to a non-naturally occurring cell, e.g., a cell that does not exist in nature, a modified cell, an engineered cell, etc., that has been obtained from an in vivo tissue or organ and is substantially free of extracellular matrix.

As used herein, the term “population of cells” refers to a plurality of cells that may be made up of any number and/or combination of homogenous or heterogeneous cell types, as described elsewhere herein. For example, a population of cells may comprise about 10%, about 20%, about 30%, about 40%, about 50%, about 60%, about 70%, about 80%, about 90%, or about 100% of a desired therapeutic cell type, e.g., hematopoietic stem or progenitor cells, immune effector cells. In certain embodiments, the cells introduced into the cell therapy vessel are isolated or purified from a population of heterogeneous cells using methods known in the art.

Illustrative examples of cell types for manufacturing in the cell therapy vessels contemplated in particular embodiments include, but are not limited to, cell lines, primary cells, stem cells, progenitor cells, and differentiated cells.

The term “stem cell” refers to a cell which is an undifferentiated cell capable of (1) long term self -renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Symmetric cell division produces two identical daughter cells. “Proliferation” or “expansion” of cells refers to symmetrically dividing cells.

As used herein, the term “progenitor” or “progenitor cells” refers to cells have the capacity to self-renew and to differentiate into more mature cells. Many progenitor cells differentiate along a single lineage, but may have quite extensive proliferative capacity.

In particular embodiments, a vessel contemplated herein comprises one or more mesodermal stem or progenitor cells. Illustrative examples of mesodermal stem or progenitor cells include, but are not limited to bone marrow stem or progenitor cells, umbilical cord stem or progenitor cells, adipose tissue derived stem or progenitor cells, hematopoietic stem or progenitor cells (HSPCs), mesenchymal stem or progenitor cells, muscle stem or progenitor cells, kidney stem or progenitor cells, osteoblast stem or progenitor cells, chondrocyte stem or progenitor cells, and the like.

In other embodiments, a vessel contemplated herein comprises one or more ectodermal stem or progenitor cells. Illustrative examples of ectodermal stem or progenitor cells include, but are not limited to neural stem or progenitor cells, retinal stem or progentior cells, skin stem or progenitor cells, and the like.

In other embodiments, a vessel contemplated herein comprises one or more endodermal stem or progenitor cells. Illustrative examples of endodermal stem or progenitor cells include, but are not limited to liver stem or progenitor cells, pancreatic stem or progenitor cells, epithelial stem or progenitor cells, and the like.

In certain embodiments, a vessel contemplated herein comprises one or more of a bone cell, osteocyte, osteoblast, adipose cell, chondrocyte, chondroblast, muscle cell, skeletal muscle cell, myoblast, myocyte, smooth muscle cell, bladder cell, bone marrow cell, central nervous system (CNS) cell, peripheral nervous system (PNS) cell, glial cell, astrocyte cell, neuron, pigment cell, epithelial cell, skin cell, endothelial cell, vascular endothelial cell, breast cell, colon cell, esophagus cell, gastrointestinal cell, stomach cell, colon cell, head cell, neck cell, gum cell, tongue cell, kidney cell, liver cell, lung cell, nasopharynx cell, ovary cell, follicular cell, cervical cell, vaginal cell, uterine cell, pancreatic cell, pancreatic parenchymal cell, pancreatic duct cell, pancreatic islet cell, prostate cell, penile cell, gonadal cell, testis cell, hematopoietic cell, lymphoid cell, or myeloid cell.

In a preferred embodiment, a vessel contemplated herein comprises a population of hematopoietic cells, e.g., hematopoietic stem cells, hematopoietic progenitor cells, immune effector cells, T cells, NKT cells, NK cells and the like.

Illustrative sources to obtain hematopoietic cells include, but are not limited to: cord blood, bone marrow, mobilized peripheral blood mononuclear cells, lymph nodes tissue, thymus tissue, tissue from a site of infection, ascites, pleural effusion, spleen tissue, and tumors.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,63 5,3 87; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells can repopulate the erythroid, neutrophil-macrophage, megakaryocyte and lymphoid hematopoietic cell pool.

Additional illustrative examples of hematopoietic stem or progenitor cells suitable for use with the vessels contemplated herein include hematopoietic cells that are CD34⁺CD38^{Lo}CD90⁺CD45^{RA-}, hematopoietic cells that are CD34⁺, CD59⁺, Thy1/CD90⁺, CD38^{Lo/-}, C-kit/CD117⁺, and Lin⁽⁻⁾, hematopoietic cells that are CD34⁺, and hematopoietic cells that are CD133⁺. In a preferred embodiment, a population of cells comprising hematopoietic cells that are CD133⁺ CD90⁺, CD133⁺ CD34⁺, or CD133⁺ CD90⁺CD34⁺ undergo one or more processing steps in a vessel contemplated herein, to manufacture a cell-based therapeutic.

In particular embodiments, a vessel contemplated herein comprises a population of hematopoietic stem and/or progenitor cells that has been, or that will be, genetically modified to express a therapeutic protein. In one embodiment, a population of hematopoietic stem and/or progenitor cells is genetically modified with a viral vector, e.g., a lentiviral vector, encoding a therapeutic protein selected from the group consisting of: a globin, a human globin, a human β-globin, a human δ-globin, a human γ-globin, a human anti-sickling β-globin, or a human β^{A-T87Q}-globin, a human β^{A-G16D/E22A/T87Q}-globin, and a human β^{A-T87Q/K95E/K120E}-globin.

In various embodiments, the hematopoietic cell is an immune effector cell. An “immune effector cell,” is any cell of the immune system that has one or more effector functions (e.g., cytotoxic cell killing activity, secretion of cytokines, induction of ADCC and/or CDC). Illustrative immune effector cells contemplated in particular embodiments are T lymphocytes, in particular cytotoxic T cells (CTLs; CD8+ T cells), TILs, and helper T cells (HTLs; CD4+ T cells). In one embodiment, immune effector cells include natural killer (NK) cells. In one embodiment, immune effector cells include natural killer T (NKT) cells.

The terms “T cell” or “T lymphocyte” are art-recognized and are intended to include thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, CD4−CD8− T cell, or any other subset of T cells. In one embodiment, the T cell is an NKT cell. Other illustrative populations of T cells suitable for use in particular embodiments include naive T cells and memory T cells.

In particular embodiments, a vessel contemplated herein comprises a population of immune effector cells that has been, or that will be, genetically modified to express a therapeutic protein, e.g., an engineered antigen receptor. In one embodiment, a population of immune effector cells is genetically modified with a viral vector, e.g., a lentiviral vector, encoding a therapeutic protein selected from the group consisting of: an engineered αβ TCR, an engineered γδ TCR, a dimerizing agent regulated immunoreceptor complex (DARIC), a chimeric antigen receptor (CAR), a bispecific T cell engager (BiTE), and zetakine receptor. In particular embodiments, the therapeutic protein is an engineered antigen receptor that binds a target antigen selected from the group consisting of: alpha folate receptor (FRa), αᵥβ₆ integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1).

### E. Adapters

The cell therapy vessels contemplated herein are designed to facilitate the manipulations of a population of cells that is manufactured into a cell-based therapy. Without wishing to be bound by any particular theory, it is contemplated in particular embodiments, that the cell therapy vessel is designed to fit into a cell therapy vessel adaptor, such that the vessel/adaptor assembly fits into commercially available centrifuges. By designing the cell therapy vessels in this way, cells may be subjected to one or more processing steps using simpler processing methods and low-cost, commonly available equipment; thereby providing a quantum improvement over existing cell therapy vessels.

In particular embodiments, a cell therapy vessel adapter is contemplated. In particular embodiments, the adaptor is made by filling a cell therapy vessel with the desired volume and making a mold or cast. In preferred embodiments, the outer dimensions of the adaptor is designed to fit into commercially available centrifuges.

In particular embodiments, a cell therapy vessel adapter comprises an inner portion that is dimensioned or shaped to receive a cell therapy vessel contemplated herein comprising a population of cells. By way of explanation, a cell therapy vessel adapter comprises an inner portion that is dimensioned or shaped to receive a cell therapy vessel having a volume of a cell therapy vessel contemplated elsewhere herein, e.g., supra. The outer portion that is dimensioned or shaped to fit into commercially available centrifuges. In preferred embodiments, a cell therapy vessel adapter comprises an inner portion that is dimensioned or shaped to receive a cell therapy vessel having a volume of a cell therapy vessel contemplated elsewhere herein; an outer portion dimensioned or shaped to fit into commercially available centrifuges; and one or more openings for intake/output lines of the vessel. In preferred embodiments, a cell therapy vessel adapter comprises an open top portion through which the cell therapy vessel can be placed into an adapter; an inner portion that is dimensioned or shaped to receive a cell therapy vessel having a volume of a cell therapy vessel contemplated elsewhere herein; an outer portion dimensioned or shaped to fit into commercially available centrifuges; and an opening at the bottom of the adapter for an intake/output line of the vessel. In another preferred embodiment, a cell therapy vessel adapter comprises an open top portion through which the cell therapy vessel can be placed into an adapter, an inner portion that is dimensioned or shaped to receive a cell therapy vessel having a volume of a cell therapy vessel contemplated elsewhere herein; an outer portion dimensioned or shaped to fit into commercially available centrifuges; an opening at the bottom of the adapter for an intake/output line of the vessel; and a groove dimensioned or shaped to receive an intake/output line that is threaded through the bottom of the adapter.

In particular embodiments, the cell therapy vessel adapter comprises one or more materials selected from the group consisting of: polyvinylchloride, polyethylene, polypropylene, and fluorinated ethylene propylene.

### F. Kits

In various embodiments, a kit for processing a population of cells is provided. In particular embodiments, a kit comprises one or more cell therapy vessels contemplated herein and one or more corresponding cell therapy vessel adapters, optionally one or more vessel clamps, and instructions for using the vessels and adapters.

In particular embodiments, a kit comprises a cell therapy vessel comprising a first intake/output line connected to the top of or upper portion of a vessel body, a vessel body comprising a rectangular portion and a conical portion, a cell collection reservoir, a second intake/output line connected to the bottom or lower portion of the cell collection reservoir, and optionally one or more intake/output ports; a corresponding cell therapy vessel adapter; and instructions for using the vessel and adapter.

In certain embodiments, a kit comprises a cell therapy vessel; a corresponding cell therapy vessel adapter comprising an open top portion through which the cell therapy vessel can be placed into an adapter, an inner portion that is dimensioned or shaped to receive a cell therapy vessel having a volume of a cell therapy vessel contemplated elsewhere herein, an outer portion dimensioned or shaped to fit into commercially available centrifuges, and an opening at the bottom of the adapter for an intake/output line of the vessel; and instructions for using the vessel and adapter.

In some embodiments, a kit comprises a cell therapy vessel; a corresponding cell therapy vessel adapter; and instructions for using the vessel and adapter to concentrate cells, perform complete medium exchanges, wash cells, prepare cells for electroporation, prepare cells for transduction, transduce cells, formulate cells for administration, and formulate cells for cryopreservation.

In preferred embodiments, a kite comprises a cell therapy vessel comprising a first intake/output line connected to the top of or upper portion of a vessel body, a vessel body comprising a rectangular portion and a conical portion, a cell collection reservoir, a second intake/output line connected to the bottom or lower portion of the cell collection reservoir, and optionally one or more intake/output ports; a corresponding cell therapy vessel adapter comprising an open top portion through which the cell therapy vessel can be placed into an adapter, an inner portion that is dimensioned or shaped to receive a cell therapy vessel having a volume of a cell therapy vessel contemplated elsewhere herein, an outer portion dimensioned or shaped to fit into commercially available centrifuges, and an opening at the bottom of the adapter for an intake/output line of the vessel; and instructions for using the vessel and adapter to concentrate cells, perform complete medium exchanges, wash cells, prepare cells for electroporation, prepare cells for transduction, transduce cells, formulate cells for administration, and formulate cells for cryopreservation.

### G. Methods of Use

In various embodiments, a method for processing a population of cells is provided. In particular embodiments, a method comprises processing and/or manufacturing a cell therapy product comprising performing one or more processing steps on a population of cells in a cell therapy vessel including, but not limited to, concentrating cells, performing complete medium exchanges, washing cells, preparing cells for electroporation, preparing cells for transduction, transducing cells, formulating cells for administration, and formulating cells for cryopreservation. Without wishing to be bound by any particular theory, it is believed that the cells therapy vessels contemplated herein provide numerous advantages over existing vessels including, but not limited to, providing a low-cost, robust, and simple solution to performing one or more cell processing steps in a single vessel using common commercial centrifuges and retrieving the processed cells by centrifugation.

In particular embodiments, a method comprises introducing a population of cells and culture medium or pharmaceutically acceptable buffer or diluent into a cell therapy vessel. The cells and medium may be introduced into the bag through an intake/output line or through a line connected to port on the vessel. In particular embodiments a solution comprising a cell culture medium and a population of therapeutic cells is introduced into a cell therapy vessel contemplated herein. In preferred embodiments, the cells are therapeutic cells that have been, or that will be, genetically modified. Once the vessel has reached the desired volume, the vessel may be placed in an adapter and loaded into a centrifuge to pellet or concentrate the population of cells and facilitate removal of the cell culture medium and/or the cells. Clamps may be used to prevent cells or media from being removed from the vessel, or to facilitate removal of media without removing the cells, or to facilitate removal of the cells without removing additional media, as desired. Fresh medium or buffer may then be introduced into the vessel to exchange media and resuspend or dilute the concentrated cells to facilitate one or more additional processing steps. In a preferred embodiment, fresh medium or buffer is introduced through an intake/output line connected to the cell collection reservoir to facilitate resuspension of the concentrated or pelleted cells.

Illustrative examples of cell culture media suitable for use in particular methods contemplated herein include, but are not limited to: QBSF-60; StemPro-34; X-VIVO 10; RPMI 1640; Clicks; AIM-V; DMEM; MEM; a-MEM; F-12; X-VIVO 15; X-VIVO 20; and any of the foregoing with added amino acids, sodium pyruvate, and vitamins, either serum-free or supplemented with an appropriate amount of serum (or plasma) or a defined set of hormones, and/or an amount of cytokine(s) sufficient for the activation, stimulation, growth, and/or expansion.

Illustrative examples of pharmaceutically acceptable buffers or diluents suitable for use in particular methods contemplated herein include, but are not limited to: HEPES, Dulbecco's phosphate buffered saline (PBS), Ringer's solution, 5% dextrose in water (D5W), and normal/physiologic saline (0.9% NaCl). The pharmaceutically acceptable buffers and/or diluents may be present in amounts sufficient to maintain a pH of the therapeutic composition of about 7. Alternatively, the therapeutic composition has a pH in a range from about 6.8 to about 7.4, e.g., 6.8, 6.9, 7.0, 7.1, 7.2, 7.3, and 7.4. In still another embodiment, the therapeutic composition has a pH of about 7.4.

In particular embodiments, a method comprises performing one or more processing steps and concentrating or pelleting the processed cells to facilitate further processing and/or retrieval of the processed cells. In particular embodiments, a cell therapy vessel comprises a population of therapeutic cells that is processed to prepare the cells for genetic modification, electroporation, transduction, cryopreservation, and/or administration.

In particular embodiments, a buffer suitable for genetic modification of the cells is introduced into a cell therapy vessel through an intake/output line or a line connected to a port. The genetic modification buffer may be added to an existing solution or used to resuspend a concentrated population of cells or cell pellet. In particular embodiments, a population of cells is washed 1, 2, 3, 4, or 5 times prior to introducing a genetic modification buffer. In some embodiments, a population of cells is washed in a wash buffer and washed 1, 2, 3, 4, or 5 times in a genetic modification buffer prior to genetic modification. In particular embodiments, following genetic modification, cells are concentrated or pelleted and fresh medium or buffer is then be introduced into the vessel to exchange mediums and resuspend or dilute the concentrated cells to facilitate one or more additional processing steps.

In particular embodiments, an electroporation buffer is introduced into a cell therapy vessel through an intake/output line or a line connected to a port. Electroporation buffer may be added to an existing solution or used to resuspend a concentrated population of cells or cell pellet. In particular embodiments, a population of cells is washed 1, 2, 3, 4, or 5 times prior to introducing an electroporation buffer. In some embodiments, a population of cells is washed in a wash buffer and washed 1, 2, 3, 4, or 5 times in an electroporation buffer prior to electroporation. In particular embodiments, following electroporation, cells are concentrated or pelleted and fresh medium or buffer is then introduced into the vessel to exchange mediums and resuspend or dilute the electroporated cells to facilitate one or more additional processing steps.

In particular embodiments, a transduction buffer is introduced into a cell therapy vessel through an intake/output line or a line connected to a port. Transduction buffer may be added to an existing solution or used to resuspend a concentrated population of cells or cell pellet. In particular embodiments, a population of cells is washed 1, 2, 3, 4, or 5 times prior to introducing a transduction buffer. In some embodiments, a population of cells is washed in a wash buffer and washed 1, 2, 3, 4, or 5 times in a transduction buffer prior to transduction. In particular embodiments, following transduction, cells are concentrated or pelleted and fresh medium or buffer is then be introduced into the vessel to exchange mediums and resuspend or dilute the concentrated cells to facilitate one or more additional processing steps.

In particular embodiments, a method comprises performing one or more processing steps and concentrating or pelleting the processed cells to facilitate formulation of the processed cells for cryopreservation and/or administration. In certain embodiments, a population of cells is formulated for cryopreservation by introducing a cryopreservation medium into the vessel. Illustrative examples of cryopreservation media suitable for use in particular methods contemplated herein include, but are not limited to: CryoStor CS10, CryoStor CS5, and CryoStor CS2. In particular embodiments, a cryopreservation medium is introduced into a cell therapy vessel through an intake/output line or a line connected to a port. A cryopreservation medium may be added to an existing solution to dilute the cryopreservation solution or added at the final concentration.

In a preferred embodiment, a solution comprising 50:50 PlasmaLyte A to CryoStor CS10 is used to resuspend a concentrated population of cells or cell pellet. Without wishing to be bound by any particular theory, one of the advantages of the vessels contemplated herein is the ability to formulate the desired number of therapeutic cells at the appropriate concentration in a formulation buffer, without requiring further dilution. In particular embodiments, a population of cells is washed 1, 2, 3, 4, or 5 times prior to formulation for cryopreservation. In some embodiments, a population of cells is washed in a wash buffer and washed 1, 2, 3, 4, or 5 times in a cryopreservation medium prior to cryopreservation. In particular embodiments, cells are formulated in a cryopreservation medium; retrieved from the vessel through an intake/output line connected to the cell collection reservoir; and frozen in a controlled rate freezer. In particular embodiments, the frozen cells are thawed and administered to a subject.

In particular embodiments, a method comprises performing one or more processing steps and concentrating or pelleting the processed cells to facilitate formulation of the processed cells for administration. In certain embodiments, a population of cells is formulated for administration by introducing a suitable medium or buffer, .e.g., PlasmaLyte, PlasmaLyte/CryoStor CS10, and the like, into the vessel. In particular embodiments, the medium or buffer is introduced into a cell therapy vessel through an intake/output line or a line connected to a port. In particular embodiments, a population of cells is washed 1, 2, 3, 4, or 5 times prior to formulation for administration. In some embodiments, a population of cells is washed in a wash buffer and washed 1, 2, 3, 4, or 5 times in a suitable medium prior to administration. In a preferred embodiment, cells are formulated in a solution comprising 50:50 PlasmaLyte A to CryoStor CS10; retrieved from the vessel through an intake/output line connected to the cell collection reservoir; and administered to a subject.

In particular embodiments, formulation with pharmaceutically-acceptable media, buffers, or diluents is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22^{nd} Edition. Edited by Loyd V. Allen Jr. Philadelphia, Pa.: Pharmaceutical Press; 2012, which is incorporated by reference herein, in its entirety.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Cell Therapy Vessel for Processing Cells

A population of cells is introduced into a cell therapy vessel through an intake/output line welded to the top of the vessel. The vessel is filled until the desired volume is reached. The vessel is placed into a cell therapy vessel adapter by threading an intake/output line attached to the bottom of the cell collection reservoir through an opening in the bottom of the adapter. The line is secured in a groove on the outside of the adapter. The adapter is placed in a standard centrifuge and the cells are centrifuged at a force and time sufficient to concentrate or pellet the cells in the cell collection reservoir. The vessel is then removed from the adapter and a clamp is fitted onto the reservoir directly above the concentrated or pelleted cells. The supernatant media will be drained out of the bag through the intake/output line at the top of the vessel. At this point, the cells can be retrieved through the intake/output line attached to the bottom of the cell collection reservoir or the cells can be resuspended or diluted in another medium or buffer for additional processing steps and/or formulated for cryopreservation and/or administration. This cycle can be repeated for any number of cell processing steps, with the ultimate step being removal of the cells from the vessel.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A cell therapy vessel comprising a first intake/output line; a body comprising a rectangular portion and a conical portion; a collection reservoir; and a second intake/output line.

2. The cell therapy vessel of claim 1, further comprising one or more ports.

3. The cell therapy vessel of claim 1, further comprising one port on the top of the vessel body and one port on the bottom of the collection reservoir.

4. The cell therapy vessel of any one of claims 1 to 3, wherein the first intake/output line is connected to the top of the vessel body.

5. The cell therapy vessel of any one of claims 1 to 4, wherein the first intake/output line is connected to the port on the top of the vessel body.

6. The cell therapy vessel of any one of claims 1 to 4, wherein the first intake/output line is welded to the top of the vessel body.

7. The cell therapy vessel of any one of claims 1 to 6, wherein the rectangular portion has a width of about 4 inches to about 6 inches.

8. The cell therapy vessel of any one of claims 1 to 7, wherein the rectangular portion has a width of about 5 inches.

9. The cell therapy vessel of any one of claims 1 to 8, wherein the conical portion has a maximum width of about 4 inches to about 6 inches and a minimum width of about 1 inch to about 0.5 inches.

10. The cell therapy vessel of any one of claims 1 to 9, wherein the conical portion has a maximum width of about 5 inches and a minimum width of about 0.75 inches.

11. The cell therapy vessel of any one of claims 1 to 10, wherein the collection reservoir has a width of about 1 inch to about 0.5 inches.

12. The cell therapy vessel of any one of claims 1 to 11, wherein the second intake/output line is connected to the bottom of the collection reservoir.

13. The cell therapy vessel of any one of claims 1 to 12, wherein the second intake/output line is connected to the port on the bottom of the collection reservoir.

14. The cell therapy vessel of any one of claims 1 to 12, wherein the second intake/output line is welded to the bottom of the collection reservoir.

15. The cell therapy vessel of any one of claims 1 to 14, wherein the length of the body of the cell therapy vessel is about 5 inches to about 7 inches.

16. The cell therapy vessel of any one of claims 1 to 15, wherein the length of the body of the cell therapy vessel is about 6 inches.

17. The cell therapy vessel of any one of claims 1 to 16, wherein the body of the cell therapy vessel has a volume of about 300 mLs to about 600 mLs.

18. The cell therapy vessel of any one of claims 1 to 17, wherein the body of the cell therapy vessel has a volume of about 300, 301, 302, 303, 304, 305, 306, 307, 308, 309, 310, 311, 312, 313, 314, 315, 316, 317,318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417,318, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517,318, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or about 600 mLs.

19. The cell therapy vessel of any one of claims 1 to 18, wherein the body of the cell therapy vessel has a volume of about 400 mLs.

20. The cell therapy vessel of any one of claims 1 to 18, wherein the body of the cell therapy vessel has a volume of about 500 mLs.

21. The cell therapy vessel of any one of claims 1 to 20, wherein the volume of the rectangular portion and the conical portion of the cell therapy vessel is about 15-fold to about 25-fold of the volume of the collection reservoir.

22. The cell therapy vessel of any one of claims 1 to 21, wherein the volume of the rectangular portion and the conical portion of the cell therapy vessel is about 20-fold of the volume of the collection reservoir.

23. The cell therapy vessel of any one of claims 1 to 20, wherein the volume of the collection reservoir is about 1% to about 10% of the volume of the rectangular portion and the conical portion of the cell therapy vessel.

24. The cell therapy vessel of any one of claims 1 to 23, wherein the volume of the collection reservoir is about 5% of the volume of the rectangular portion and the conical portion of the cell therapy vessel.

25. The cell therapy vessel of any one of claims 1 to 24, wherein the cell therapy vessel is comprised of one or more materials selected from the group consisting of:
polyvinylchloride, polyethylene, polypropylene, and fluorinated ethylene propylene.

26. A cell therapy vessel comprising:
(a) a first intake/output line fused to the top of the vessel;
(b) a rectangular portion;
(c) a conical portion;
(d) a cell collection reservoir; and
(e) a second intake/output line fused to the bottom of the cell collection reservoir.

27. The cell therapy vessel of claim 26, further comprising one or more ports.

28. The cell therapy vessel of claim 26 or claim 27, wherein the rectangular portion has a width of about 4 inches to about 6 inches.

29. The cell therapy vessel of any one of claims 26 to 28, wherein the rectangular portion has a width of about 5 inches.

30. The cell therapy vessel of any one of claims 26 to 29, wherein the conical portion has a maximum width of about 4 inches to about 6 inches and a minimum width of about 1 inch to about 0.5 inches.

31. The cell therapy vessel of any one of claims 26 to 30, wherein the conical portion has a maximum width of about 5 inches and a minimum width of about 0.75 inches.

32. The cell therapy vessel of any one of claims 26 to 31, wherein the collection reservoir has a width of about 1 inch to about 0.5 inches.

33. The cell therapy vessel of any one of claims 26 to 32, wherein the length of the body of the cell therapy vessel is about 5 inches to about 7 inches.

34. The cell therapy vessel of any one of claims 26 to 33, wherein the length of the body of the cell therapy vessel is about 6 inches.

35. The cell therapy vessel of any one of claims 26 to 34, wherein the body of the cell therapy vessel has a volume of about 300 mLs to about 600 mLs.

36. The cell therapy vessel of any one of claims 26 to 35, wherein the body of the cell therapy vessel has a volume of about 300, 301, 302, 303, 304, 305, 306, 307, 308, 309, 310, 311, 312, 313, 314, 315, 316, 317,318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417,318, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517,318, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or about 600 mLs.

37. The cell therapy vessel of any one of claims 26 to 36, wherein the body of the cell therapy vessel has a volume of about 400 mLs.

38. The cell therapy vessel of any one of claims 26 to 36, wherein the body of the cell therapy vessel has a volume of about 500 mLs.

39. The cell therapy vessel of any one of claims 26 to 38, wherein the volume of the rectangular portion and the conical portion of the cell therapy vessel is about 15-fold to about 25-fold of the volume of the collection reservoir.

40. The cell therapy vessel of any one of claims 26 to 39, wherein the volume of the rectangular portion and the conical portion of the cell therapy vessel is about 20-fold of the volume of the collection reservoir.

41. The cell therapy vessel of any one of claims 26 to 38, wherein the volume of the collection reservoir is about 1% to about 10% of the volume of the rectangular portion and the conical portion of the cell therapy vessel.

42. The cell therapy vessel of any one of claims 26 to 41, wherein the volume of the collection reservoir is about 5% of the volume of the rectangular portion and the conical portion of the cell therapy vessel.

43. The cell therapy vessel of any one of claims 26 to 42, wherein the cell therapy vessel is a bag comprised of one or more materials selected from the group consisting of:
polyvinylchloride, polyethylene, polypropylene, and fluorinated ethylene propylene.

44. The cell therapy vessel of any one of claims 1 to 43, wherein the cell therapy vessel contains a population of cells.

45. The cell therapy vessel of claim 44, wherein the population of cells is genetically modified.

46. The cell therapy vessel of claim 44, wherein the population of cells comprises one or more genome edits.

47. The cell therapy vessel of claim 44, wherein the population of cells comprises a gene therapy vector.

48. The cell therapy vessel of claim 47, wherein the gene therapy vector encodes a therapeutic protein.

49. The cell therapy vessel of claim 47 or claim 48, wherein the gene therapy vector encodes a chimeric antigen receptor (CAR) or engineered T cell receptor (TCR).

50. The cell therapy vessel of claim 44, wherein the population of cells is obtained from peripheral blood mononuclear cells, bone marrow, lymph nodes tissue, cord blood, thymus issue, tissue from a site of infection, ascites, pleural effusion, spleen tissue, or tumors.

51. The cell therapy vessel of claim 44, wherein the population of cells comprises hematopoietic stem or progenitor cells.

52. The cell therapy vessel of claim 44, wherein the population of cells comprises CD34⁺ cells.

53. The cell therapy vessel of claim 44, wherein the population of cells comprises lymphocytes.

54. The cell therapy vessel of claim 44, wherein the population of cells comprises T cells.

55. The cell therapy vessel of claim 44, wherein the population of cells comprises CD3⁺, CD4⁺, and/or CD8⁺ T cells.

56. The cell therapy vessel of claim 44, wherein the population of cells comprises effector cells.

57. The cell therapy vessel of claim 44, wherein the population of cells comprises cytotoxic T lymphocytes (CTLs), tumor infiltrating lymphocytes (TILs), or helper T cells.

58. The cell therapy vessel of claim 44, wherein the population of cells comprises natural killer (NK) cells or natural killer T (NKT) cells.

59. The cell therapy vessel of any one of claims 26 to 58, wherein the cell therapy vessel is a bag that comprises one or more materials selected from the group consisting of:
polyvinylchloride, polyethylene, polypropylene, and fluorinated ethylene propylene.

60. The cell therapy vessel of any one of claims 1 to 59, wherein the cell therapy vessel is endotoxin free.

61. A cell therapy vessel adapter comprising:
(a) an inner portion shaped to receive the cell therapy vessel of any one of claims 1 to 60;
(b) an outer portion shaped to fit into a centrifuge;
wherein the outer portion further comprises an opening for the second intake/output line of the cell therapy vessel and a groove to receive the second intake/output line.

62. The cell therapy vessel adapter of claim 61, wherein the cell therapy vessel adapter comprises one or more materials selected from the group consisting of:
polyvinylchloride, polyethylene, polypropylene, and fluorinated ethylene propylene.

63. A kit comprising: (a) a cell therapy vessel according to any one of claims 1 to 60, and (b) a cell therapy vessel adapter of claim 61 or claim 62.

64. The kit of claim 63, further comprising one or more clamps.

65. A method for processing a cell therapy product comprising introducing a population of cells into the cell therapy vessel of any one of claims 1 to 60; placing the cell therapy vessel into the cell therapy vessel adapter of claim 61 or claim 62, and centrifuging the population of cells under conditions sufficient to concentrate the population of cells in the cell collection reservoir of the cell therapy vessel.

66. The method of claim 65, wherein processing comprises performing one or more processing steps selected from the group consisting of: concentrating the cells, exchanging cell culture medium or buffer, washing the cells, genetically modifying the cells, preparing the cells for electroporation, preparing the cells for transduction, transducing the cells, formulating the cells for administration, or formulating the cells for cryopreservation.

67. The method of claim 65 or claim 66, wherein the population of cells is genetically modified.

68. The method of any one of claims 65 to 67, wherein the population of cells is concentrated by centrifugation after one or more processing steps.

69. The method of any one of claims 65 to 68, where processing comprises introducing one or more materials into the cell therapy vessel through the one or more ports or the first or second intake/output line.

70. The method of any one of claims 65 to 68, where processing comprises removing one or more materials from the cell therapy vessel through the one or more ports or the first or second intake/output line.

71. The method of any one of claims 65 to 69, wherein the population of cells is introduced to the cell therapy vessel through the first intake/output line.

72. The method of any one of claims 65 to 71, wherein the population of cells is removed from the cell therapy vessel through the first intake/output line.

73. A method for manufacturing a cell-based therapy comprising introducing a population of cells into the cell therapy vessel of any one of claims 1 to 60; placing the cell therapy vessel into the cell therapy vessel adapter of claim 61 or claim 62, and centrifuging the population of cells under conditions sufficient to concentrate the population of cells in the cell collection reservoir of the cell therapy vessel.

74. The method of claim 73, wherein the cell-based therapy is an adoptive cellular therapy, a cell-based gene therapy, or a genome-edited cell therapy.

75. The method of claim 73 or claim 74, wherein the population of cells is genetically modified before introduction into the vessel.

76. The method of claim 73 or claim 74, wherein the population of cells is genetically modified in the vessel.

77. The method of any one of claims 74 to 76, wherein the cell-based therapy is CAR T cell therapy.

78. The method of any one of claims 74 to 76, wherein the cell-based therapy is a gene therapy for hemoglobinopathies.

79. The method of any one of claims 74 to 76, wherein the cell-based therapy is a gene therapy for adrenoleukodystrophies.

80. The method of any one of claims 74 to 76, wherein the cell-based therapy is a genome-edited cell therapy for a cancer.

81. The method of claim of any one of claims 73 to 80, wherein processing comprises performing one or more processing steps selected from the group consisting of: concentrating the cells, exchanging cell culture medium or buffer, washing the cells, genetically modifying the cells, preparing the cells for electroporation, preparing the cells for transduction, transducing the cells, formulating the cells for administration, or formulating the cells for cryopreservation.

82. The method of claim of any one of claims 73 to 81, wherein the population of cells is concentrated by centrifugation after one or more processing steps.

83. The method of any one of claims 73 to 82, where processing comprises introducing one or more materials into the cell therapy vessel through the one or more ports or the first or second intake/output line.

84. The method of any one of claims 73 to 83, where processing comprises removing one or more materials from the cell therapy vessel through the one or more ports or the first or second intake/output line.

85. The method of any one of claims 73 to 84, wherein the population of cells is introduced to the cell therapy vessel through the first intake/output line.

86. The method of any one of claims 73 to 85, wherein the population of cells is removed from the cell therapy vessel through the first intake/output line.
